(19) United States
(12) Patent Application Publication
(10) Pub. No.: US 2013/0131506 A1
(43) Pub. Date: May 23, 2013

(54) INTRAVENOUS INFILTRATION DETECTION

(71) Applicant: MURRAY M. POLLACK, PHOENIX, AZ (US)

(72) Inventor: MURRAY M. POLLACK, PHOENIX, AZ (US)

(21) Appl. No.: 13/299,603

(22) Filed: Nov. 18, 2011

(51) Int. Cl. A61B 6/00; A61B 5/01

(52) U.S. Cl. 600/431; 600/549

(57) ABSTRACT

A process and apparatus system for detecting an intravenous infiltration. A separate infiltration detecting solution containing an intravenously administerable infiltration indicator is intravenously administered singly or on a periodic basis and a qualitative and/or quantitative assessment of the indicator in tissue proximate and/or distal to the site of intravenous administration of a biological fluid is carried out.

### BACKGROUND OF THE INVENTION

Intravenous (IV) infusion as a delivery means of a biological fluid to a patient is a common clinical procedure in medical settings. In the United States, approximately 80% of hospitalized patients receive IV therapy. In general, a needle or cannula is inserted into a peripheral vein and connected to an IV bag containing a biological fluid by means of a flexible connector tube. The biological fluid can be mere saline solution and/or contain any of the numerous therapeutic agents and/or nutrients administerable by IV infusion.

At times, and due to various causes, the IV fluid infiltrates nonvascular tissue proximate to the site of injection or infusion, adversely affecting the surrounding tissue and disrupting the therapeutic schedule. Further, the infiltration can permeate tissue distal to the site of IV infusion, such as in the vicinity of the venous drainage system of the IV infusion. The IV solution can accumulate in the skin and subcutaneous tissue, possibly damaging the skin and tissues and preventing critical medication and/or nutrients from reaching the venous system. Thus, several complications can result from an infiltration, including extravasation, tissue necrosis, phlebitis, venous inflammation, reduced therapy delivery and others.

Most studies on infiltrations have shown an incidence of infiltration of approximately 30 to 50%. Such infiltrations are difficult to detect, especially at an early stage of infiltration. To date, the techniques to detect infiltrations primarily rely on clinical methods, which include visual inspection of the IV site, visual inspection of the connector tube for blood return, and visual and tactile examination of the skin and tissue surrounding and proximate to the IV injection site for such factors as tissue pressure, color, edema, turgor and temperature. Some practitioners rely on manual infusion of fluid through the IV system using a syringe followed by tactile examination by hand of the IV site. To date, these clinical methods for attempting to detect extravasations have proven to be unreliable, especially at an early stage of infiltration and in young children where there can be large dressings around the IV site to protect the IV from the child but which would interfere with visual and tactile examination. Further, these clinical methods are often in error with infiltrations that are in progress or are moderately symptomatic.

Various nonclinical approaches to detect infiltrations have been proposed. So far these nonclinical methods have been largely unsuccessful. For example, resistance to flow and/or increased pressure within the IV connector tube is monitored by a detector emitting an alarm when flow resistance or pressure increases above a threshold value. In general, IV occlusion devices in IV pump systems require pressure or resistance changes within the IV tubing, requiring large pressure changes in the extravascular tissue near the insertion locus for detecting a moderate or severe infiltration. Devices which monitor tissue characteristics have been proposed. These include devices monitoring changes in optical characteristics, electromagnetic radiation characteristics and temperature occurring in surrounding tissues. Systems using such devices have not proven their usefulness and as a result are not in widespread use. In particular, these latter techniques have not been found to be useful detection devices for early stage infiltrations because the changes required by these devices require substantial amounts of infiltrate or tissue reaction.

U.S. Pat. No. 6,375,624 describes an IV infiltration detection device based on microwave radiometry and involves placement of a microwave antenna near the injection needle for sensing variations in subsurface tissue temperature. The antenna is connected to a radiometer for amplifying the microwave emissions received by the antenna. Tissue and fluid temperatures are monitored and processed through an alarm circuit which is activated in the event of an infiltration. A surface temperature sensor can be used instead of the antenna. This reference illustrates a monitoring system sensing changes in temperature in tissue surrounding the point of injection into the venous system. This patent recognizes that in clinical situations, the injected fluid temperature and the tissue temperature at the site of injection may be very close, preventing an accurate assessment of infiltration. For overcoming this difficulty, alternative embodiments are described in this reference where either the injected fluid or the surrounding tissue at the area of injection are heated or cooled to provide a temperature differential sufficient for infiltration detection. These embodiments necessitate heating and/or cooling means to be attached directly to the connector tube and/or the patient. Constant heating and/or cooling would be needed for maintaining the desired tissue/fluid temperature differential.

US 2002/0172323 describes systems and methods for detecting infiltration of contrast media used with contrast-enhanced computed tomography (CT). An imaging-based method using very low dose x-rays detects the absorption profile of the lumen or blood vessel and of the surrounding tissue area. An alternate embodiment using ultrasound instead of x-rays is also described. Thus, this reference attempts to indirectly visualize an infiltration.

U.S. Pat. No. 7,184,820 describes tissue monitoring devices for detecting harmful conditions in tissues, including infiltrations and extravasations. Non-invasive physiological measurements are taken by sensors capable of detecting tissue conditions and being incorporated into a film barrier dressing. Bio-impedence sensing, infrared sensing, temperature sensing, photonics and others are described as sensing technologies, to be used alone or in combination. The overall system is highly complex in nature, requiring sophisticated device elements.

U.S. Pat. No. 7,591,792 relates to electromagnetic sensors used for detecting fluids, such as infiltrations and extravasations, and other foreign materials in tissues. The invention of this patent lies in the structure of the sensor.

U.S. Pat. No. 4,647,281 describes a microwave antennae system for detecting changes in tissue temperature as indicative of an infiltration. One antenna device is placed at the area of possible infiltration with a second reference antenna placed at a distance there from to provide a reference or base tissue temperature. Obviously, this system is only operable if the fluid being injected is of sufficient temperature difference from the tissue at the point of injection.

### SUMMARY OF THE INVENTION

It is an object of the present invention to provide a new process for indicating the occurrence of an infiltration or extravasation during the intravenous (IV) administration of a biological fluid to a patient.

Another object of the present invention is to provide a novel apparatus system useful for indicating the occurrence of an infiltration or extravasation during the IV administration of a biological fluid to a patient.

Still another object of the present invention is to provide a new process and novel apparatus system for carrying out the process in which an administered detector means comprising an indicator of infiltration means is used for detecting the occurrence of an infiltration or extravasation during the IV administration of a biological fluid to a patient.

A further object of the present invention is to provide an IV infiltration or extravasation detection means in which an extrinsic indicator is used which can be observed by visual or tactile examination of tissue adjacent to or distal to the site of IV injection.

Still another object of the present invention is to provide an IV infiltration or extravasation detection means in which an extrinsic indicator is used which can be detected by an apparatus or apparatus system, partly positioned adjacent to and/or distal to the site of IV injection.

Still a further object of the present invention is to provide an IV infiltration or extravasation detection process and apparatus system which does not require the direct heating and/or cooling of the therapeutically administered biological fluid and/or tissue adjacent the site of IV injection.

Another object of the present invention is to provide an IV infiltration or extravasation detection process and apparatus system suitable for use with pediatric patients in a clinical setting within or without a hospital.

A further object of the present invention is to provide an IV infiltration or extravasation detection process and apparatus system for detection of infiltrations or extravasations at an early stage of IV infusion, prior to the time when the infiltration or extravasation might be detectable by clinical examination.

Still another object of this invention is to provide an IV infiltration or extravasation detection process and apparatus system for detection of infiltrations or extravasations that have reached a stage of infiltration causing clinical changes consistent with infiltrations or extravasations.

Other objects of the present invention are to reduce the harm, such as chemical burns, sloughs, amputations and non-delivery of medications, that can occur due to infiltrations or extravasations into the tissue adjacent or distal to a point of IV infusion, and to provide a safe patient environment.

Other objects of the present invention will be apparent to the skilled artisan from the detailed description of the invention as set forth herein.

Therefore, in accordance with the present invention it has now been found that the occurrence of an IV infiltration or extravasation can be determined by singly or periodically injecting a detector solution comprising an indicator of infiltration through an IV positioned cannula through which an IV administerable biological fluid is being intravenously administered and qualitatively and/or quantitatively testing tissue adjacent or downstream from the site of IV injection for presence of the indicator.

In one preferred embodiment of the present invention, the indicator is temperature and the detector solution is a heated or cooled liquid periodically injected through an IV positioned cannula through which an IV administerable biological fluid is being intravenously administered and testing for an infiltration or an extravasation is carried out by tactile examination of tissue surrounding, adjacent to, near or downstream to the site of IV injection. In another preferred embodiment of the present invention, the detector solution is a cold liquid periodically injected through an IV positioned cannula through which an IV administered biological fluid is administered and testing for an infiltration or an extravasation is carried out by a temperature sensing apparatus, including a sensor element positioned in one or more arrays surrounding, adjacent to, near and/or downstream to the site of IV injection. In a more preferred embodiment of the instant invention, the detector solution is a cold IV administerable liquid and the temperature sensing device comprises one or more arrays comprising thermistors.

In another preferred embodiment of the present invention, the detector solution containing an indicator material is periodically injected through an IV positioned cannula through which an IV administerable biological fluid is intravenously administered and testing for an infiltration or an extravasation is carried out by testing tissue surrounding, adjacent to, near or downstream of the site of IV injection for the presence of the indicator. In one aspect of this embodiment of the invention, the indicator can be visually observed. In a second aspect of this embodiment of the invention, the presence of the indicator can be determined using a sensing apparatus for sensing the presence of the indicator. In this second aspect of the present invention, the indicator sensing device qualitatively and/or quantitatively measures the indicator in tissue surrounding, adjacent to, near or downstream to the site of IV injection.

Further aspects of the present invention involve apparatus systems including a receptacle containing an IV administerable biological fluid, a receptacle containing an IV infiltration or extravasation detecting solution, IV injector means through which the biological fluid is injected into a vein of a patient, a first connector means through which the biological solution flows from its receptacle to an entrance to the injector means and a second connector means through which the IV infiltration or extravasation detecting solution flows from its receptacle to an entrance into the injector means. In this embodiment of the invention, the injector means is a cannula or needle carrying a multi-channel port at the entrance thereof and the receptacle containing the IV infiltration or extravasation detecting solution is a syringe or an IV fluid bag. In one preferred embodiment of this aspect of the invention, a temperature sensing device is positioned surrounding, adjacent to, near and/or downstream of the point of IV injection. In another version of this apparatus aspect of the invention, the IV infiltration or extravasation detecting solution of the inventive apparatus system contains an indicator substance and then the apparatus system includes a sensing device for qualitatively and/or quantitatively sensing the presence of the indicator substance in tissue proximate or distal to the site of IV injection. In preferred embodiments of this aspect of the invention, the indicator substance is a colored dye and the sensing device is a colorimeter.

In another preferred embodiment of the present invention, the indicator is an emitted ray or particle provided by the indicator substance and the sensing device senses the emitted ray or particle. The sensing device can be a fluorometer or a radiation sensor.

In certain embodiments of the present invention, an array of sensors or detectors is placed on tissue areas lateral, circumferential and/or distal to the IV infusion cannula, and/or in the venous drainage area of the IV infusion at known distances from the inserted cannula. In certain preferred apparatus systems of the present invention, an alarm processor is included as part of the overall apparatus system. The alarm processor is automatically triggered when the indicator sensing device registers a predetermined level of indicator in the sensed tissue, thereby alerting personnel that an infiltration or extravasation has occurred. In a more preferred embodiment of this aspect of the invention, the alarm is situated in a circuit which can automatically slow or stop the infusion of the IV solution when the sensing device registers a predetermined level of indicator in the sensed tissue.

Other embodiments and variations of the invention will be apparent to the skilled artisan from the detailed description of the invention hereinafter.

### BRIEF DESCRIPTION OF THE DRAWINGS

FIG. 1 illustrates a first embodiment of the present invention in which a detector solution is administered when desired, from a receptacle holding the same, to the patient through a multichannel port and valve assembly.

FIG. 2 is a cross-sectional view of a multichannel port and valve assembly as can be used in the embodiments of FIGS. 1 and 3.

FIG. 3 illustrates a second embodiment of the present invention in which a detector solution is administered when desired, by means of a syringe holding the same, to the patient through a multichannel port and valve assembly.

FIG. 4 is a schematic block diagram illustrating an embodiment of the present invention in which arrays of sensors are positioned on a limb of a patient and a signal analyzer and alarm processor are used to automatically monitor the patient for determining whether an infiltration or extravasation has occurred and, if so, to automatically slow down or stop the flow of the therapeutic IV biological solution to the patient.

### DETAILED DESCRIPTION OF THE INVENTION

The present invention provides a method and means for allowing medical personnel to detect the occurrence of an infiltration or extravasation during intravenous (IV) infusion in a clinical setting. IV infusion is a standard and often preferred method for administering therapeutic agents and nutrients directly into the blood stream of a patient. Herein, the term “therapeutic agent” is inclusive of all medicinal and nutritional substances which are administered via IV infusion in a continuous manner to a patient, as opposed to substances which are administered in a single injectable bolus. Of course, the IV infusion need not contain a therapeutic agent. The IV administerable therapeutic agent, for example, is inclusive of continuously administered anesthetics, continuously administered diagnostic agents such as contrast agents, continuously administered antibiotics and other drugs, continuously administered vitamins and other nutrients which at times are considered drugs, and the like.

The term “infiltration” as used herein means leakage of an IV administered solution from the vein into which the solution is administered proximate to the point of IV administration. The term “extravasation” means the same as an infiltration or, in a narrower sense, an infiltration of an agent which can adversely affect tissue into which the solution leaks, such as an IV administered agent which can cause tissue necrosis. Even though an infiltration occurs at or near the point of injection of the IV solution into the vein, the leaked fluid not only permeates the tissue at and near the point of injection, but, depending somewhat on its physical and chemical characteristics, can migrate downstream of the site of IV injection into the venous drainage system.

The adverse affects of an infiltration or extravasation are twofold. First, at least a portion of the patient-needed therapeutic agent, which most often has been designed for rapid onset of biological activity through its IV administration, is now found within the extravascular tissue proximate and distal to the site of injection, where its biological availability has most likely been significantly compromised. Second, one or more substances within the IV infusion solution, once leaked into extravascular tissue, may be capable of causing any of a number of different adverse events in the extravascular tissue, such as minor to moderate to severe inflammation, tissue necrosis and so on.

An early infiltration is most often caused by one of two conditions. First, the patient due to age could be suffering from a general deterioration of peripheral veins allowing seepage into the extravascular tissue. Second, the inserted cannula could have punctured the vein and thereby provided a route for seepage of IV fluid into surrounding tissue.

Malposition of an IV catheter or cannula can occur at initial insertion into the peripheral vein, most often in the hand or arm. For example, the cannula could be forced completely through the vein resulting in direct injection of the IV fluid into tissue surrounding the vein. At times, the aperture at the injection terminus of the cannula ends up partially within and partially outside the selected vein, this time resulting in partial delivery of the IV solution into the vein and partial delivery of the IV solution into tissue surrounding the vein. Sometimes the cannula punctures the vein at more than one location, resulting in seepage of the IV solution from the vein into adjacent tissue. Further, the cannula may be moved laterally during its insertion into the vein, causing the puncture aperture into the vein to become larger than the diameter of the inserted part of the cannula, again resulting in leakage around the cannula into surrounding tissue. In any event, these are merely examples of the type of problem that may result during initial placement of the catheter and which can cause an infiltration or extravasation. Certainly, other problems can result in initial malposition of the cannula in the body of the patient with a resulting infiltration or extravasation. Similarly, the cannula can become malpositioned during its prolonged time of placement in the patient. For example, even the pressure resulting on the cannula in the patient's body could produce a malposition, or simple movements of the patient's extremity could produce a malposition. In both cases, an infiltration or extravasation could result during the course of IV administration. A malposition of the cannula during prolonged use is especially of concern with pediatric patients who can have a tendency to move about. The present invention not only provides an efficient way to check for an infiltration or extravasation caused by an initial malposition, but also provides a means for periodically checking for an infiltration or extravasation caused by a malposition of the cannula occurring during the course of IV fluid administration, which can continue over the course of several days.

The detector solution involved with the present invention is desirably administered to the patient as close as possible to the site of cannula insertion into the vein. For reasons described hereinafter in detail, it is highly desirable, but at times not necessary, to avoid to the extent reasonable significant admixture of the detector solution with the IV biological infusion solution. Especially, this is important where a quantitative assessment of an infiltration or extravasation is desired. Certainly, minor admixture can be tolerated, especially when only a qualitative assessment of an infiltration or extravasation is being carried out. Thus, in general, it is best to dilute the detector solution as little as possible, or to a predefined extent, especially if a quantitative measurement of infiltration or extravasation is to be ascertained.

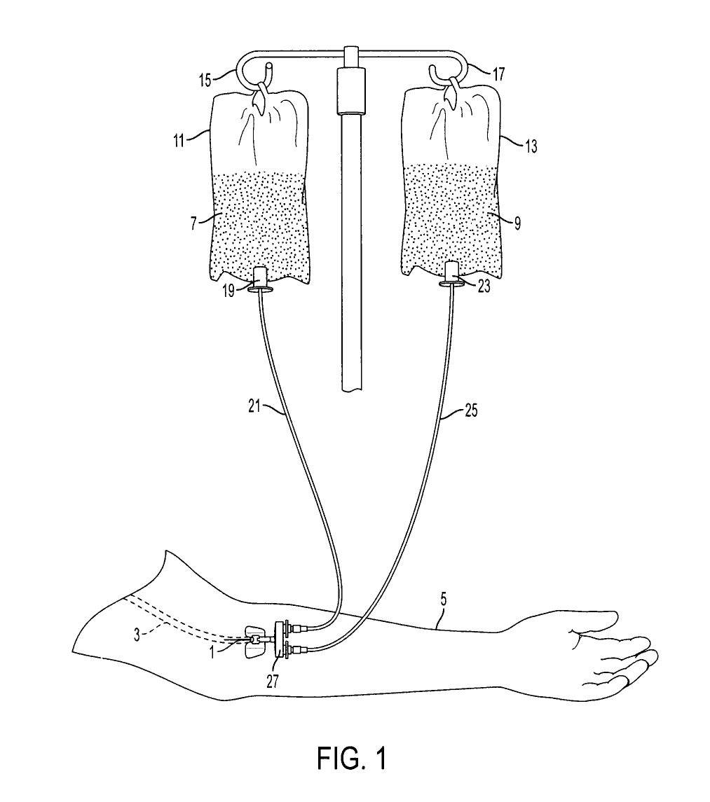

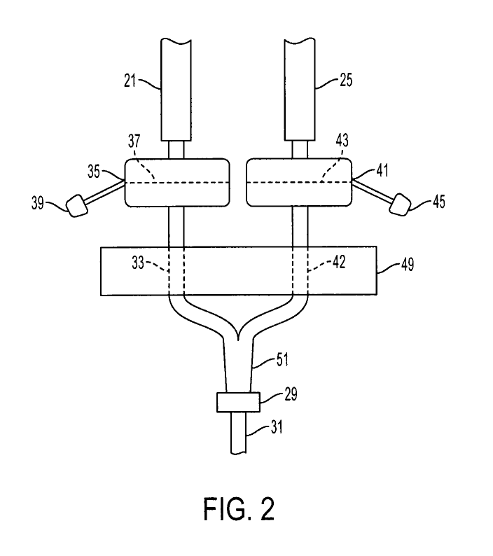

In FIG. 1, one embodiment of the placement of the detector supply apparatus in relationship to the biological solution infusion apparatus is illustrated. Cannula 1 has been inserted into vein 3 positioned in arm 5. Biological IV fluid 7 is found within IV bag 11, which in turn is held above arm 5 by holding device 15. Similarly, detector solution 9 has been placed within IV fluid bag 13, also supported above the patient's arm, this time by bag holding apparatus 17. Drip regulators 19 and 23 allow slow ascendancy of the biological IV fluid and the detector solution, respectively, essentially filling connector tubes 21 and 25 with those solutions, respectively. The two connector tubes are connected to multichannel port and valve apparatus 27, shown in detail in FIG. 2, for delivery, when desired, of the biological IV fluid and of the infiltration or extravasation detector solution to the patient.

FIG. 2 is a cross-sectional view of the multichannel port and valve apparatus shown as element 27 in FIG. 1. Connector conduits 21 and 25 are attached to unidirectional valves 35 and 41, respectively. These two valves are shown as stopcock valves in FIG. 2, containing valve plates 37 and 43, which are shown in the closed position but rotate 90 degrees within the valve housings, by manipulation of handles 39 and 45 to allow passage of solutions 7 and 9 into multichannel port 49. In FIG. 2, multichannel port 49 contains two internal passages 33 and 42. Depending upon manipulation of the two unidirectional valves, one or both of the biological infusion solution and the detector solution flows through the multichannel port into short connecting conduit 51 and through connector 29 into the end 31 of the cannula 1. Various types of one-way valves can be employed in the practice of this invention, as will be realized by the skilled artisan. Also, the multichannel port can contain more than two channel flow passages depending upon the number of solutions to be delivered through the multichannel port to the patient.

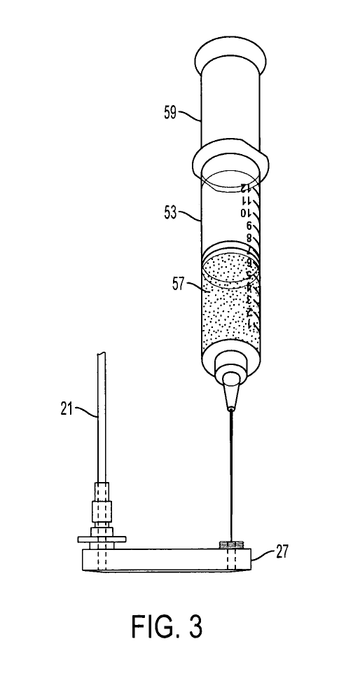

FIG. 3 depicts an alternate detector solution delivery embodiment to deliver the detector solution to the multichannel port. In this embodiment syringe 53 contains detector solution 57 and is shown with its needle inserted into multichannel port 27. Depression of syringe plunger 59 ejects the detector solution into and through the multiport channel into the patient, not shown. Alternatively, the detector solution can be directly injected through the IV or the IV tubing valves. In this alternative embodiment, the IV infusion solution would be stopped during a bolus injection of the detector.

In general, the apparatus delivery system of FIG. 1 is most useful when the detector solution can remain under ambient conditions for a length of time, for example, for the duration of the IV therapy or for a significant segment thereof, for example, the embodiment of the present invention in which a colored dye solution is used as the indicator. The apparatus system of FIG. 3 is most useful when the detector solution should not remain under ambient conditions for a significant length of time, for example, the embodiment of the present invention in which a cold solution is used as the indicator. This is not to say that a cold detector solution can not be delivered using the delivery system of FIG. 1 and that a colored dye solution can not be delivered by use of the delivery system of FIG. 3. In most instances, the detector solution delivery apparatus systems depicted in FIGS. 1 and 3 are interchangeable in the practice of the present invention. For example, with a cold detector solution, a cooling jacket, not shown, could surround receptacle 13, or other cooling means could be employed.

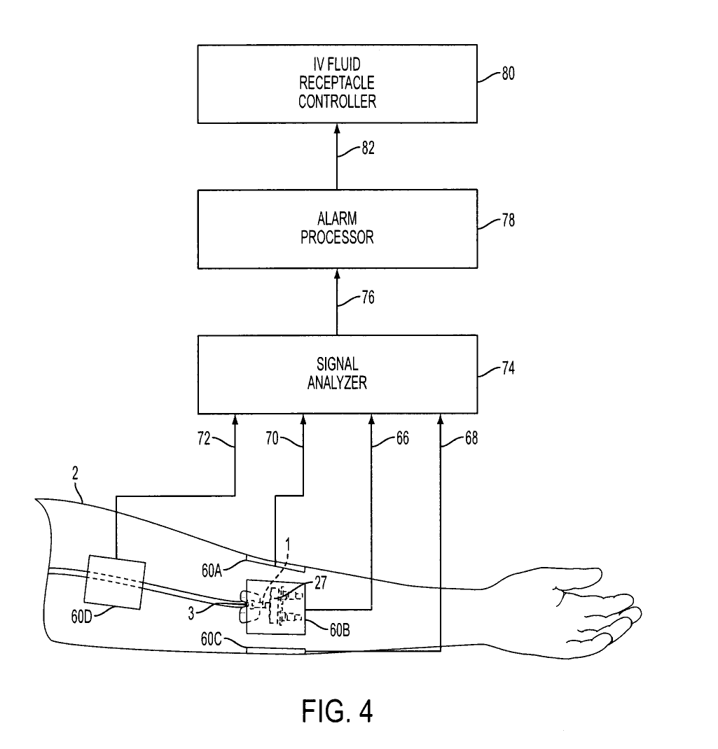

FIG. 4 is a plan view of an apparatus system of the present invention depicting arrays of sensors attached via an adhesive not shown to the limb 2 into which the IV biological solution and detector solution are being administered. Sensing arrays 60A, 60B, 60C and 60D detect the identical indicator but are placed at different sensing areas of the patient's limb. Sensing array 60B is situated directly over and surrounding the site of injection, while sensing arrays 60A and 60C are positioned laterally to the injection site and surrounding skin areas lateral to the site of injection. Sensing array 60D is positioned over a venous drainage area for the site of injection. Information, such as temperature, color intensity, radioactivity, fluorescence and the like from the sensing detectors is transmitted by means of electrical connectors 66, 68, 70 and 72 to signal analyzer 74, which processes the received information for determining whether an infiltration or extravasation has occurred, and in preferred embodiments, processes the information for determining the degree of the infiltration or extravasation. In turn, the analyzed information is optionally forwarded via electrical connector 76 to alarm processor 78, if desired. This alarm processor uses a microprocessor (not shown) to read the signals from the signal analyzer and where desired information such as flow rate for the IV biological solution and/or flow rate of the detector solution or where the detector is temperature, the temperature of the detector solution. The alarm processor has been programmed for threshold sensing values, which can be stored in a table by the microprocessor. The proper threshold value may be selected by an algorithm based on the quantity or quality of the delivered detection solution, or another indicator characteristic such as temperature of the detector solution. When the alarm processor senses that the predetermined threshold value has been exceeded, it then determines the magnitude thereof and depending on that calculation declares an infiltration or extravasation. The alarm processor can be programmed to emit an audible signal at that time, plus it can be programmed to slow down or stop the flow of the IV biological fluid to the patient, as is known in the art. For example, the alarm processor can stop the flow of fluid from electrically monitored IV fluid receptacle controller 80 by transmitting a stop signal thereto through electrical line 82.

The detector solution comprises an indicator of infiltration such as temperature, or can be an indicator of infiltration substance. The indicator substance is any substance capable of detection visually, through tactile examination or by one or more sensors positioned proximate or distal to the site of IV infusion. The indicator substance may be the material actually detected or it may provide the material actually detected, such as a particle or ray.

In the most preferred embodiment of the present invention, the detector solution is a cooled or cold IV administerable solution. The cooled detector solution is at a temperature which provides a sufficient temperature differential between it and normal body temperature for detection purposes. For example, the cooled detector solution is maintained at a temperature so that the temperature of the detector solution as it enters the IV infusion site is at a temperature of about 40 to 70 degrees Fahrenheit. With reference to FIG. 1, IV fluid bag 13 containing detector solution 9 can be maintained at a desired temperature within the above range by a cooling jacket, not shown. In that instance, a small amount of detector solution within connector tube 25 may need to be infused prior to the infusion of the cooled solution. Of course, with the use of the system shown by FIG. 3, it is only necessary to have the detector solution within syringe 53 cooled to the desired temperature immediately prior to injection.

Although it is theoretically possible to use a temperature dependent detector solution above the normal body temperature, in practice this is not desirable since an IV injected solution even a few degrees above normal body temperature can cause tissue damage.

In the most preferred embodiment of the invention involving a cooled detector solution, arrays of temperature sensors are positioned proximate and distal to the site of IV infusion. These temperature sensors are preferably comprising thermistors, although other temperature sensors can be used, such as thermocouples. The thermistor can be embedded within a thin plastic sheet which in turn carries an adhesive for attachment to human skin. This type of adhesive is well known in the medical arts. Further, in clinical environments where temperature sensors are not readily available, the clinician can rely upon tactile examination of tissue proximate and distal to the IV infusion site for detecting an infiltration or extravasation based on a temperature differential between normal body temperature and the temperature of the detector solution. However, tactile examination is not a preferred embodiment, especially where it is desirable to analyze the degree of any infiltration or extravasation. Preferably, a known quantity of a cold IV infusion at a set temperature is administered and then the thermistors measure tissue temperatures proximate and/or at a set distance distal to the site of IV infusion, and all of this information is fed into the microprocessor of the analyzer, which then based on previously determined mathematical models, determines whether an infiltration or extravasation has occurred and the degree thereof.

Other indicators comprise various kinds of dyes. The dyes are those which can be used at present in clinical settings as well as such dyes as are developed in the future.

The dye can be a colored substance, the color of which can be detected by the human eye and/or by colorimetric sensors. Preferably, the detector solution contains an indicator dye of sufficient color intensity so that if an infiltration or extravasation occurs, the colorimetric sensors can relay degrees of color intensity to the colorimetric analyzer for determining the degree of any detected infiltration or extravasation, in accordance with a predetermined mathematical model. The colorimetric analyzer can be programmed to process change in color, hence dye intensity, over time. Computations can include the previously observed and expected change produced by a defined amount of the dye-containing detector solution without an infiltration or extravasation as compared with the actual observations during an IV infusion coupled with periodic infusion of the defined amount of the dye colored detector solution. Examples of a colored substance type dye usable in the practice of the present invention are indocyanine green and Evans blue.

Also, the dye can be a fluorescent dye. Often, the fluorescent dye absorbs light invisible to the human eye, such as ultraviolet light, and emits light in the visible region to the human eye. Examples of fluorescent dyes usable in the practice of the present invention are derivatives of xanthene (i.e. fluorescein), cyanine (i.e. indocarbocyanine) and oxazine (i.e. Nile red). In this embodiment of the invention, the detector solution containing the substance that fluoresces is exposed to, for example, ultraviolet light, as it passes through the connector tube near the site of IV infusion. Then, detection sensors positioned in the sensing arrays measure the amount of fluorescence emitted by any resulting infiltration or extravasation. The degree of an infiltration or extravasation is determined based on appearance of the dye in non-venous areas, and may be quantified based on the known amount of fluorescence produced by a given quantity of the detector solution over time as compared with the analysis of the fluorometer measured values. Of course, where necessary, mere visual examination of fluorescence in tissues proximate and/or distal to the site of IV infusion would be indicative of an infiltration or extravasation.

Another indicator substance is a radioactive material providing radiation. Various radioactive substances are currently used in medical diagnostic procedures, and would be usable in the practice of the present invention. Preferably, the radioactive substance has an extremely short half life, of about 15 minutes to several hours. The emitted radiation is in the form of particles or rays, the quantity of which can be measured using arrays of radiation measuring devices coupled to an analyzer. This embodiment of the invention can provide a very accurate assessment of the degree of infiltration or extravasation, when such occurs by either qualitatively documenting radioactivity in the nonvascular areas in the vicinity of the intravenous site, or the extent of the infiltration or extravasation can be quantified by measuring the radioactivity using an appropriate detector knowing the quantity of the radioactive indicator administered, and knowing the half life of the radioactive indicator. A known amount of radiation is provided by a certain quantity of periodically administered detector solution. The precise amount of infiltrate can be determined by analysis of the radiation measured by the arrays of radiation sensors. Examples of potential radiation producing materials usable in the practice of this invention are bismuth-213 and technetium-99m.

The quantity of detector solution administered can vary significantly depending upon the weight of the patient and the indicator substance employed. As an example, for an average adult, the amount of cold detector solution administered is about 3 to 10 ml, while for pediatric patients, the cold detector solution is administered in a quantity of about 0.5 to 3 ml, with the smaller quantities being suitable for infants. For other indicator substances disclosed herein, the amount of administered detector solution is generally about the same volume as above, but can be dependent on the concentration of the indicator. It is necessary to administer a quantity of detector solution, if an infiltration or extravasation has occurred, sufficient to permeate tissues surrounding and/or distal to the site of IV infusion.

The detector solution is administered periodically based on the clinician's judgment. Most often, the detector solution is administered shortly after the initiation of IV therapy, say about 1 to 15 minutes after initiation of IV therapy. Thereafter, the detector solution can be administered about every 4 to 12 hours, or more or less often based on the condition of the patient. For example, if tissue puffiness is observed near the site of IV infusion, then the detector solution is administered to check for a possible infiltration or extravasation. Similarly, elderly patients having reduced vascular wall elasticity or restless young patients may require a more frequent administration schedule of detector solution.

The skilled artisan will readily appreciate that other indicator substances can be used in the practice of the present invention, in addition to those discussed above. For example, other luminescent nanoparticles could be used with selection of appropriate sensor arrays.

Other variations of the invention will be apparent to the skilled artisan upon review and understanding of the invention set forth herein.

What is claimed is:

1. A process for determining whether an infiltration has been caused in a patient by an intravenously administered biological fluid, which comprises during substantially continuous administration of the biological fluid, intravenously administering to the patient an infiltration detecting solution containing an indicator of infiltration and determining the presence of the indicator in patient tissue proximate and/or distal to a site of administration of the biological fluid into the patient.

2. The process of claim 1 wherein the infiltration detecting solution is administered once following the start of administration of the biological fluid.

3. The process of claim 1 wherein the infiltration detecting solution is administered periodically following the start of administration of the biological fluid.

4. The process of claim 1 wherein the administration of the biological fluid is stopped during the administration of the infiltration detecting solution.

5. The process of claim 1 wherein at least one indicator of infiltration sensor is positioned over patient tissue proximate or distal to the site of administration of the biological fluid into the patient.

6. The process of claim 1 comprising the step of qualitatively assessing the presence of the indicator in tissue of the patient.

7. The process of claim 1 comprising the step of quantitatively assessing the presence and quantity of the indicator in tissue of the patient.

8. The process of claim 1 wherein one or more array(s) of indicator of infiltration sensors is (are) positioned over patient tissue proximate and/or distal to the site of administration of the biological fluid into the patient.

9. The process of claim 1 wherein the infiltration detecting solution is administered from an intravenous fluid bag containing the infiltration detecting solution.

10. The process of claim 1 wherein the infiltration detecting solution is administered from a syringe containing the infiltration detecting solution.

11. The process of claim 1 wherein the infiltration detecting solution is a cooled intravenously administerable solution.

12. The process of claim 11 wherein the infiltration detecting solution is at a temperature of about 40 to 70 degrees Fahrenheit as administered into the patient.

13. The process of claim 11 wherein at least one array of temperature sensors is positioned over patient tissue proximate and/or distal to the site of administration of the biological fluid into the patient.

14. The process of claim 13 wherein the temperature sensors comprise thermistors.

15. The process of claim 1 wherein the indicator of infiltration is a dye.

16. The process of claim 15 wherein the dye is a colored dye.

17. The process of claim 15 wherein the dye is a fluorescent dye.

18. The process of claim 1 wherein the indicator of infiltration is radiation.

19. The process of claim 8 wherein signals from the indicator sensors are analyzed by a signal analyzer.

20. The process of claim 19 wherein data produced by the signal analyzer is processed by an alarm processor programmed, when a threshold signal reading is reached, to automatically decrease the flow of the biological fluid to the patient.

21. The process of claim 1 wherein the indicator of infiltration is radiation.

22. An apparatus for detecting an infiltration in tissue of a patient being intravenously administered a biological fluid, the apparatus comprising:
a receptacle means containing an infiltration detecting solution;
a multichannel port and valve means through which intravenously administered fluid is inserted into the patient; and
a fluid transport element having one terminus thereof connected to the receptacle means and another terminus thereof connected to a first channel of the multichannel port and valve means.

23. The apparatus of claim 22 wherein the receptacle means is an intravenous fluid bag.

24. The apparatus of claim 22 wherein the receptacle means is a syringe.

25. The apparatus of claim 22 also comprising:
an intravenous fluid bag containing the biological fluid and
a second fluid transport element having one terminus thereof connected to the intravenous fluid bag containing the biological fluid and having another terminus thereof connected to another channel of the multichannel port and valve means.

26. The apparatus of claim 22 wherein the multichannel port and valve element comprises a separate valve associated with each channel thereof used for administering a fluid to the patient.

27. The apparatus of claim 22 also comprising at least one indicator of infiltration sensing means.

28. The apparatus of claim 22 also comprising at least one array of indicator of infiltration sensors.

29. The apparatus of claim 28 wherein the sensors are temperature sensors.

30. The apparatus of claim 29 wherein the sensors comprise thermistors.

31. The apparatus of claim 28 wherein the sensors are dye sensors.

32. The apparatus of claim 31 wherein the sensors are color sensors.

33. The apparatus of claim 31 wherein the sensors are fluorescence sensors.

34. The apparatus of claim 27 also comprising a signal analyzer for analyzing data generated by said sensing means.

35. The apparatus of claim 34 also comprising an alarm processor for processing data generated by said signal analyzer.

36. The apparatus of claim 35 also comprising an intravenous fluid receptacle controller regulated by said alarm processor.

37. The apparatus of claim 34 wherein the signal analyzer is a temperature analyzer.

38. The apparatus of claim 34 wherein the signal analyzer is a dye intensity analyzer.

39. The apparatus of claim 34 wherein the signal analyzer is a radiation analyzer.

40. The apparatus of claim 28 wherein the sensors are radiation sensors.

41. The process of claim 1 wherein the biological fluid does not contain a therapeutic agent or a nutrient.

42. The process of claim 1 wherein the biological fluid contains a therapeutic agent or a nutrient.
